(19) United States
(12) Patent Application Publication
(10) Pub. No.: US 2015/0374878 A1
(43) Pub. Date: Dec. 31, 2015

(54) ANGIOGENIC DEVICES FOR WOUND CARE

(71) Applicant: AKESO BIOMEDICAL, INC., Waltham, MA (US)

(72) Inventors: Andrew J. Carter, Stow, MA (US); Simon F. Williams, Sherborn, MA (US)

(21) Appl. No.: 14/752,078

(22) Filed: Jun. 26, 2015

Related U.S. Application Data
(60) Provisional application No. 62/018,232, filed on Jun. 27, 2014.

(51) Int. Cl. A61L 15/44; A61L 15/26; A61L 15/64; A61L 15/20; A61L 15/42

(52) U.S. Cl. CPC A61L 15/44; A61L 15/20; A61L 15/425; A61L 15/64; A61L 15/26; A61L 2300/21; A61L 2300/412

(57) ABSTRACT

Devices, such as dressing and implants, for treatment of chronic wounds comprising controlled delivery systems for butyric acid or salts, polymers, or derivatives thereof are provided. These devices are particularly useful when it is desirable to promote angiogenesis in a chronic wound. In the preferred embodiment, the implants are resorbable, provide a temporary scaffold for the in-growth of cells, tissues, and blood vessels to help regenerate the extracellular matrix, and deliver butyric acid to the chronic wound. The dressings and implants may also contain one or more antibiotics to treat or prevent infection in the wound, and/or inhibitors of proteases to modulate protease activity in the wound.

### FIELD OF THE INVENTION

The present invention generally relates to devices used in wound care, and in particular to the healing of wounds, such as chronic wounds, where angiogenesis is desirable.

### BACKGROUND OF THE INVENTION

It has been estimated that about 26 million patients suffer from chronic wounds each year. Chronic wounds include diabetic foot ulcers, venous stasis ulcers, pressure ulcers, burns, and surgical wounds. Those at highest risk for developing chronic wounds include patients with diabetes, disabilities, and the elderly. These patients suffer not only from the physical pain of the wound, but also from stress and a poor quality of life.

Standard treatment for chronic wounds usually involves cleaning the wound, debriding the wound, and applying a dressing to maintain a moist tissue environment conducive to healing. In many cases, treatment also includes the use of antibiotics since chronic wounds are also frequently infected. Antibiotics may be administered systemically and/or by using a dressing containing an antibiotic. Clinicians will also try to eliminate underlying factors that cause the formation of chronic wounds.

Unfortunately, a significant number of patients with chronic wounds will still not be healed after 3 months, 6 months, or even after one year of treatment. In the worst cases, amputation may be necessary, and elderly patients may even develop sepsis and die.

There are a number of reasons why chronic wounds are difficult to heal. One reason is the lack of new blood vessel formation that is necessary to support newly deposited tissue during the wound healing process. The formation of new blood vessels is known as angiogenesis, and is a necessary process in the healing of chronic wounds to promote blood flow into the wound and support the metabolic activity of new tissue. The lack of angiogenesis in chronic wounds is thought to be due in part to the absence of growth factors, and may also be related to the inability of the body to form a new extracellular matrix (ECM) that can host new blood vessels.

Research on the formation of new ECM in chronic wounds has led to the development, for example, of products like PROMOGRAN® by Systagenix, which incorporates oxidized regenerated cellulose (ORC). The ORC inhibits proteases in chronic wounds that are considered to be detrimental to the formation of new ECM in order to improve wound healing.

Clinicians have also experimented with the use of autologous wound healing factors, derived from patient's blood, to improve wound healing. For example, the topical application of platelet-derived growth factor (PDGF) has been investigated in the clinic And researchers have also evaluated the use of platelet-rich plasma (PRP), derived from autologous blood, in the healing of chronic wounds. McNeil Pharmaceutical has also introduced a recombinant PDGF product, REGRANEX® Gel, to heal diabetic ulcers. Unfortunately, in 2008 the manufacturer added a warning to the product noting that an increased incidence of mortality secondary to malignancy was observed when patients were treated with three or more tubes of the REGRANEX® Gel in a post-market retrospective cohort study.

The use of butyric acid to promote angiogenesis in a chronic wound is highly desirable. However, butyric acid has a very short half-life in serum of about 2 minutes, and the compound needs to be continuously present in order to exert a biological effect (see U.S. Pat. No. 5,858,365 to Faller). Therefore continuous administration of butyric acid to a wound or systemically administering butyric acid is not a practical treatment approach for promoting angiogenesis in a chronic wound.

There have been several disclosures of the use of butyric acid to promote angiogenesis. U.S. Pat. No. 8,541,027 to Wright et al. discloses fixation devices, including sutures, surgical arrows, staples, darts, bolts, screws, buttons, anchors, nails, rivets or barbed devices impregnated with butyric acid or salts thereof in order to promote angiogenesis. Leek et al., “Augmentation of tendon healing with butyric acid-impregnated sutures”, Am. J Sports Med, 40:1762-1771 (2012) disclosed the use of butyric acid impregnated sutures to improve tendon healing. U.S. Pat. No. 5,858,365 to Faller discloses the treatment of wounds using butyric acid salts and derivatives, such as the treatment of a leg ulcer by intravenous administration of arginine butyrate continuously for 20 days.

However the disclosures of U.S. Pat. No. 8,541,027 to Wright et al., Leek et al. Am. J. Sports Med., 40:1762-1771 (2012) and U.S. Pat. No. 5,858,365 to Faller, does not provide devices or methods for treatment of chronic wounds that incorporate scaffolds to allow the regeneration of the extracellular matrix (“ECM”) or for the controlled release of butyric acid to chronic wounds.

Therefore there is a need for devices, such as implants and dressings, for the controlled delivery of butyric acid to chronic wounds. There is also a need for devices that regenerate the ECM in a chronic wound, and preferably encourage formation and vascularization of an ECM.

It is therefore an object of the present invention to provide improved devices, such as dressings and implants, for treating a chronic wound.

It is still a further object of the present invention to provide processes for making such devices.

It is still another object of the present invention to provide improved methods to treat chronic wounds.

### SUMMARY OF THE INVENTION

Devices, such as dressings and implants, for treatment of chronic wounds that include controlled delivery systems for butyric acid or salts, polymers, or derivatives thereof, are described herein. These dressings and implants are particularly useful when it is desirable to promote angiogenesis in a chronic wound. In the preferred embodiment, the implants are resorbable, providing a temporary scaffold for the in-growth of cells, tissues, and blood vessels to help regenerate the extracellular matrix, and deliver butyric acid to the chronic wound. The dressings and implants may also comprise antibiotics to treat or prevent infection in the wound, and/or inhibitors of proteases to modulate protease activity in the wound.

The devices allow delivery of butyric acid to the wound in a controlled manner, and over a prolonged period of time. The devices are preferably made from polymeric compositions, more preferably resorbable polymers, including silk.

### BRIEF DESCRIPTION OF THE DRAWINGS

FIG. 1 is a cross-sectional view of one embodiment 10 of a coaxial needle 12 that can be used to make core-sheath fibers, showing a vertical port 14 for the delivery of a core material and a horizontal port 16 for the delivery of a sheath material from a second needle 18. Solutions of the core and sheath materials are passed simultaneously through the needle 12 and precipitated in a non-solvent to form core-sheath fibers. The coaxial needle 12 may be used to prepare core-sheath fibers for the controlled release of butyric acid or salts, polymers, or derivatives thereof. Luer lock fittings 20, 22 make the device easy to attach to a supply of material.

### DETAILED DESCRIPTION OF THE INVENTION

Methods and that allow the controlled release of butyric acid or salts, polymers, or derivatives thereof (including any isotopes thereof) for the treatment of chronic wounds, have been developed. The devices may be dressings that temporarily cover a wound (and may be in contact with a wound), but are subsequently removed from the wound, or implants that are applied to or into the wound, but are not removed. In both cases, the devices are configured to continually dose the wound with butyric acid to promote angiogenesis and healing of the wound. When the device is an implant, it is preferably a resorbable implant that provides a temporary scaffold to promote regeneration of the ECM. The scaffolds allow and/or encourage in-growth of cells, tissues, and blood vessels to help regenerate the ECM, in addition to delivering butyric acid to the chronic wound to stimulate and promote angiogenesis. The implants are preferably made from resorbable polymeric materials. Preferably the scaffolds of the resorbable implants are made from proteins, such as silk or collagen, but can be other biodegradable polymers such as polyhydroxy acids like polyglycolic acid, polylactic acid or copolymers thereof, polyanhydrides, poly 3-hydroxybutyrate or poly 4-hydroxybutyrate, polysaccharides such as alginate, celluloses, or other proteins such as collagen. Butyric acid or salts, polymers, or derivatives thereof may be incorporated in solid form, as particles or incorporated into nano or microparticles into the resorbable scaffold to produce a controlled release system for these therapeutics agents, or these therapeutic agents may be incorporated into another resorbable material and combined with the scaffold.

When the device is a dressing, the dressing may be made from a non-resorbable material or a resorbable material. Butyric acid or salts, polymers, or derivatives thereof may be incorporated directly into the dressing to produce a controlled release system, or these agents may be incorporated into another material and combined with the dressing.

The devices continuously deliver therapeutic quantities of butyric acid to a wound to promote angiogenesis. This eliminates the need to administer multiple oral or injectable doses of butyric acid, or continuously apply butyric acid to a wound dressing in order to maintain a therapeutic dose in the wound. The implants provide a temporary scaffold for the in-growth of cells, tissues, and blood vessels to help regenerate the extracellular matrix, as well as deliver butyric acid in a controlled manner to the wound to promote angiogenesis. Some of the devices comprise antibiotics to treat or prevent infection, and/or protease inhibitors to modulate protease activity in the wound. In a preferred embodiment, the device is an implant comprising a temporary resorbable porous protein scaffold, such as silk or collagen, that encourages the in-growth of cells, tissues, and blood vessels to assist in regenerating the ECM, and promoting angiogenesis by releasing butyric acid or salts, polymers, or derivatives thereof into the chronic wound.

### I. Definitions

“Angiogenesis” as used herein generally refers to the formation and differentiation of blood vessels.

“Bioactive agent” is used herein to refer to therapeutic, prophylactic, and/or diagnostic agents. It includes without limitation physiologically or pharmacologically active substances that act locally or systemically in the body. “Bioactive agent” includes a single such agent and is also intended to include a plurality.

“Biocompatible” as generally used herein means the biological response to the material or device being appropriate for the device's intended application in vivo. Any metabolites of these materials should also be biocompatible.

“Blend” as generally used herein means a physical combination of different polymers, as opposed to a copolymer comprised of two or more different monomers.

“Chronic wounds” is used herein to refer to wounds that have not healed in three months.

“Controlled release” as generally used herein refers to time dependent release of bioactive agents. It generally refers to the sustained release of bioactive agents to prolong the therapeutic action of the bioactive agent, and preferably to maintain the concentration of the bioactive agent in a therapeutic window.

“Resorbable” as generally used herein means the material is broken down in the body and eventually eliminated from the body. The terms “resorbable”, “degradable”, “erodible”, and “absorbable” are used somewhat interchangeably in the literature in the field, with or without the prefix “bio”. Herein, these terms will be used interchangeably to describe material broken down and absorbed or eliminated by the body within five years, whether degradation is due mainly to hydrolysis or mediated by metabolic processes.

### II. Compositions

Methods have been developed to produce devices that allow the controlled release of butyric acid or salts, polymers, or derivatives thereof for the treatment of chronic wounds. Suitable devices include dressings and implants. The devices may be used for the treatment of wounds, including chronic wounds such as venous stasis ulcers, diabetic ulcers, pressure ulcers, burns, and surgical wounds.

In one embodiment, dressings may be applied to the chronic wounds, and release butyric acid or salts, polymers, or derivatives thereof into the wound in a controlled manner to stimulate angiogenesis. These dressings may subsequently be removed, and if necessary replaced with new dressings.

In another embodiment, the devices may be implants that deliver butyric acid or salts, polymers or derivatives thereof into the wound in a controlled manner to stimulate angiogenesis. These implants are not substantially removed from the wound (although the implants may include protective barriers, for example, to control moisture in the wound, that may be removed from the surface of the implant). Preferably the implants are temporary scaffolds that are incorporated into the body, and allow cell, tissue, and blood vessel in-growth as they resorb.

A. Biomaterials

The devices described herein are preferably produced from polymeric compositions. When the devices are dressings, the devices can be made from permanent (i.e. non-resorbable) or resorbable polymeric compositions. When the devices are implants, the devices are preferably made from resorbable polymeric compositions.

1. Polymers

a. Non-Resorbable Polymers

Permanent polymers that may be used to prepare the dressings include, but are not limited to, poly(ethylene), poly(propylene), poly(tetrafluoroethylene), poly(methacrylates), poly(methylmethacrylate), ethylene-co-vinylacetate, poly(dimethylsiloxane), poly(ether-urethanes), poly(ethylene terephthalate), nylon, polyurethane, poly(sulphone), poly(aryletherketone), poly(ethyleneoxide), poly(ethyleneoxide-co-propyleneoxide), poly(vinylpyrrolidine), and poly(vinylalcohol).

Resorbable Polymers

Resorbable polymers that may be used to prepare the devices (dressings or implants) include, but are not limited to, proteins, including silk, collagen (including Types I to V and mixtures thereof), gelatin, and proteins comprising one or more of the following amino acids: alanine, arginine, asparagine, aspartic acid, cysteine, glutamine, glutamic acid, glycine, histidine, isoleucine, lysine, methionine, phenylalanine, proline, serine, threonine, tryptophan, tyrosine and valine; polysaccharides, including alginate, amylose, celluloses such as carboxymethylcellulose, chitin, chitosan, cyclodextrin, dextran, dextrin, gellan, glucan, hemicellulose, hyaluronic acid, derivatized hyaluronic acid, oxidized cellulose, pectin, pullulan, sepharose, xanthan and xylan; resorbable polyesters, including resorbable polyesters made from hydroxy acids (including resorbable polyesters like poly(lactides), poly(glycolides), poly(lactide-co-glycolides), poly(lactic acid), poly(glycolic acid), poly(lactic acid-co-glycolic acid), poly(dioxanones), polycaprolactones and polyesters with one or more of the following monomeric units: glycolic, lactic; trimethylene carbonate, p-dioxanone, or s-caprolactone), and resorbable polyesters made from diols and diacids; polycarbonates; tyrosine polycarbonates; polyamides (including synthetic and natural polyamides, polypeptides, and poly(amino acids)); polyesteramides; poly(alkylene alkylates); polyethers (such as polyethylene glycol, PEG, and polyethylene oxide, PEO); polyvinyl pyrrolidones or PVP; polyurethanes; polyetheresters; polyacetals; polycyanoacrylates; poly(oxyethylene)/poly(oxypropylene) copolymers; polyacetals, polyketals; polyphosphates; (phosphorous-containing) polymers; polyphosphoesters; polyalkylene oxalates; polyalkylene succinates; poly(maleic acids); biocompatible copolymers (including block copolymers or random copolymers); and hydrophilic or water soluble polymers, such as polyethylene glycol, (PEG) or polyvinyl pyrrolidone (PVP), with blocks of other biocompatible or biodegradable polymers, for example, poly(lactide), poly(lactide-co-glycolide), or polycaprolactone or combinations thereof. Resorbable polymers also include cross-linked polymers, and include, for example, cross-linked collagen, as well as functionalized polymers. Particularly preferred resorbable polymers are silk and resorbable polyesters.

Biological materials may also be used to prepare the implants and dressings. Examples of biological materials include allogenic or xenographic tissues such as acellular dermal matrix materials, cell-seeded dermal matrix material or cell-seeded resorbable polymers, and small intestine submucosa.

The polymeric materials may be blended to produce the dressings and implants.

Although the implants are preferably made from resorbable polymeric compositions, the implants may incorporate permanent materials that do not remain in or on the body. For example, a device comprising a resorbable implant may also incorporate a permanent material, such as film, to control the moisture content of the wound or prevent infection. Although a resorbable implant is left in the wound to resorb, the permanent material is eventually removed.

B. Additives

The properties of the polymeric compositions used to make the devices may be modified by the incorporation of certain biocompatible additives. Preferably, for resorbable implants, the additives are also resorbable. The additives are preferably incorporated during a compounding process, or by using a solution-based process.

In one embodiment, the additives are nucleating agents and/or plasticizers, added in a sufficient quantity to produce the desired outcome, such as improvement in mechanical properties or modification of controlled release. Nucleating agents may be incorporated in amounts of up to 20% by weight to increase the rate of crystallization of the polymeric compositions. Plasticizers may be incorporated into the polymeric compositions in amounts of up to 50% by weight, more preferably up to 30% by weight, to improve mechanical properties and controlled release rates. For example, plasticizers may be incorporated to increase the flexibility of a polymeric composition. Examples of plasticizers that may be incorporated into the devices include, but are not limited to, citrate esters, glycerol, glycerin, glycerol triacetate, dodecanol, and natural oils. In a preferred embodiment, glycerin or glycerol may be incorporated into polymeric compositions of silk.

Other additives, such as dyes, pH indicators, and diagnostic compounds, may also be incorporated into the polymeric compositions.

The agents to be released can also be formulated to alter release, for example, by providing as particles, aggregates, or incorporated into excipient such as the polymers described above to form nanoparticles or microparticles, using known techniques such as milling, precipitation, solvent evaporation or spray drying.

C. Therapeutic, Prophylactic or Diagnostic Agents

In addition to incorporating butyric acid or salts, polymers, or derivatives thereof into the devices to stimulate angiogenesis, other therapeutic, prophylactic or diagnostic agents may also be incorporated. These agents may be added during the preparation of the polymeric compositions, or may be added later to the devices. They may be added before, during or at the same time as the butyric acid or salts, polymers, or derivatives are incorporated. The agents may be added by using aqueous or solvent-based processes or melt-based processes.

Agents may be proteins, peptides, lipids, lipoprotein, nucleic acid or nucleoprotein, polysaccharide, metals, small molecules, or combinations thereof. Examples of agents that can be incorporated into the devices include, but are not limited to, growth factors, inhibitors of matrix metalloproteinases (MMPs), antibiotics (including silver particles), biofilm inhibitors, vitamins, anti-inflammatory drugs, lipids, steroids, hormones, antibodies, signaling ligands, amniotic membrane materials, anti-septic agents, analgesics, anesthetics, molecules that promote the formation of ECM, vascularization, and wound healing. Particularly preferred antibiotics include bacitracin, neomycin, polymixin B, zinc, fusidic acid, gentamicin, mafenide acetate, metronidazole, minocycline, mupirocin, nitrofurazone, polymixin, retapamulin, rifampin, silver particles, silver sulfadiazine, sulfacetamide, vancomycin, and combinations thereof.

D. Angiogenic Agents or Angiogenesis Prodrugs

The devices described herein incorporate butyric acid or salts, polymers, or derivatives thereof as angiogenic agents or agents that are converted in vivo into angiogenesis agents (i.e. angiogenesis prodrugs). In a preferred embodiment, the angiogenic agents are butyric acid salts, polymers or derivatives. Butyric acid may be incorporated into the devices, but has an unpleasant odor. Salts of butyric acid include, but are not limited to, sodium, lithium, potassium, ammonium, calcium, iron, magnesium, manganese, silver, zinc, barium, copper, iron, quaternary ammonium, and salts with amino acids such as arginine, lysine, and histidine. Sodium, silver, lithium, ammonium, potassium, arginine and lysine are preferred salts. The silver salt of butyric acid is particularly preferred because of the antibiotic properties of silver ions. Salts of butyric acid may also be formed with polymers, for example, butyric acid salts of polylysine.

Derivatives of butyric acid include esters, anhydrides, amides, orthoesters, and thioesters of butyric acid. Particularly preferred derivatives include monobutyrin, dibutyrin and tributyrin, and other fatty acid glycerides of butyric acid. Other suitable derivatives of butyric acid include succinamide, butyramide, and ethyl butyrate. Polymers of butyric acid include butyric acid esters of poly(alcohols), such as polyvinylalcohol.

“Butyric acid or salts, polymers, or derivatives thereof” as used herein also includes compounds with the carbon, hydrogen or oxygen isotopes (e.g. C¹², C¹³, C¹⁴, O¹⁶O¹⁸, deuterium, and tritium) both in their natural isotopic ratios, or enriched for one or more isotopes of carbon, hydrogen or oxygen. Deuterium isotopes of butyric acid are particularly preferred examples of enriched isotopes.

### III. Wound Healing Devices and Methods of Manufacturing

Methods have been developed to produce devices that allow the controlled release of butyric acid or salts, polymers, or derivatives thereof for the treatment of chronic wounds. In one embodiment, the methods described herein may be used to incorporate butyric acid or salts, polymers, or derivatives thereof in one step into controlled release dressings and implants for treatment of chronic wounds. In another embodiment, the methods may be used to incorporate butyric acid or salts, polymers, or derivatives thereof into controlled release forms, such as fibers, films, spheres, gels, and foams. These forms may then be incorporated into devices, such as dressings and implants, for treatment of chronic wounds.

A. Controlled Release Wound Healing Devices Manufactured from Fibers

The devices may contain fibers. The fibers may be manufactured, for example, by solution or melt-based processes, including monofilament and multifilament fiber spinning. These fibers may subsequently be knit or woven into devices, or multifilament fiber may be further processed to make non-woven devices. Butyric acid or salts, polymers, or derivatives thereof may be incorporated into these fibers during melt-extrusion or solvent spinning by blending with the permanent or resorbable polymers described above prior to spinning For example, butyric acid or salts, polymers, or derivatives thereof may be dissolved or suspended in aqueous solutions of water-soluble polymers or solvent-soluble polymers prior to spinning, or the butyric acid or salts, polymers, or derivatives thereof may be blended with the permanent or resorbable polymers and melt-extruded. These monofilament and multifilament fibers may then be incorporated as components into devices for wound healing. For example, these fibers may be knit or woven to form devices, or the multifilament fibers may be processed into non-woven wound healing devices, for example, by crimping, carding, and needling processes. In one preferred embodiment, resorbable polyesters are blended or dissolved in solvent with butyric acid or salts, polymers, or derivatives thereof, and spun to form monofilament and multifilament fibers comprising butyric acid or salts, polymers, or derivatives thereof. These fibers may be knit, weaved, or crimped, carded and needled to make fiber-based dressings and implants that can deliver butyric acid or salts, polymers, or derivatives thereof in a controlled manner for wound healing. In another preferred embodiment, aqueous solutions of silk may be combined with butyric acid or salts, polymers, or derivatives thereof, and solution spun to form fibers comprising butyric acid or salts, polymers, or derivatives thereof. The fibers may be knit, weaved or crimped, carded and needled to make silk devices for the controlled release of butyric acid or salts, polymers, or derivatives thereof for wound healing. Suitable aqueous silk solutions may be prepared, for example, by boiling silk cocoons with sodium carbonate to degum the silk and reduce its molecular weight, dissolving the silk in lithium bromide to form an aqueous silk solution, dialyzing the solution, and if necessary concentrating the silk solution.

Fiber-based wound healing devices that can deliver butyric acid or salts, polymers, or derivatives thereof in a controlled manner for wound healing may also be manufactured by dry spinning, electrospinning, centrifugal spinning, melt-blowing, spun bond processing, or combinations thereof. For example, aqueous or solvent solutions of the permanent or resorbable polymers described above containing butyric acid or salts, polymers, or derivatives thereof, may be dry spun, electro spun, or centrifugally spun to form devices for healing wounds. In a preferred embodiment, aqueous silk solutions prepared as described above may be combined with butyric acid or salts, polymers, or derivatives thereof, and electrospun or centrifugally spun to form devices for healing wounds. In another preferred embodiment, resorbable polyesters may be blended with butyric acid or salts, polymers, or derivatives thereof, and melt-blown, spun-bonded, or dry spun to form devices for healing wounds.

The fiber-based wound healing devices described herein may also be prepared without butyric acid or salts, polymers, or derivatives thereof incorporated, and subsequently treated to incorporate these compounds. For example, fiber-based devices of monofilaments and multifilaments, or electrospun, centrifugally spun, spun bond, dry spun or melt-blown non-wovens, may be coated with butyric acid or salts, polymers, or derivatives thereof by dipping, painting, immersing, or spray-coating these fiber-based devices. In a preferred embodiment, devices comprising butyric acid or salts, polymers, or derivatives thereof may be prepared by coating, dipping, immersing, or painting silk-based wound healing constructs with solutions of butyric acid or salts, polymers, or derivatives thereof.

In a preferred embodiment, the fiber-based implants described herein have pores that will allow the in-growth of blood vessels.

B. Controlled Release Wound Healing Devices Manufactured from Films

The devices may contain films made from the permanent and/or resorbable polymers described above. The films may be prepared by melt-extrusion, compression molding, or solvent casting, and may also, if desired, be perforated. Butyric acid or salts, polymers, or derivatives thereof may be blended with the permanent and resorbable polymers, and melt-extruded or compression molded to form films, or these compounds may be dissolved in solutions with the permanent and resorbable polymers, and cast to form films Alternatively, films with or without butyric acid or salts, polymers, or derivatives thereof may be coated, dipped, or sprayed with solutions of butyric acid or salts, polymers, or derivatives thereof. In a preferred embodiment, butyric acid or salts, polymers, or derivatives thereof may be combined with aqueous silk solutions, and cast to form films comprising one or more of these compounds for wound healing. These cast films may be perforated to allow the in-growth of blood vessels if the films are used as implants. In another preferred embodiment, resorbable polyesters may be blended with butyric acid or salts, polymers, or derivatives thereof, and melt-extruded to form film-devices for wound healing.

C. Controlled Release Wound Healing Devices Manufactured from Foams and Sponges

The devices may contain foams, including open and closed-cell foams, sponges, and other porous forms. These foams may be produced, for example, by phase-separation, melt-foaming, and particulate leaching methods. In phase-separation, a solvent system for the butyric acid or salts, polymers, or derivatives thereof and the permanent or resorbable polymer may be used to form a solution, which is cast to form a film The film is frozen to precipitate the polymer, and the solvent sublimated using, for example, a lyophilizer, to form a phase separated porous polymeric foam comprising butyric acid or salts, polymers, or derivatives thereof. In a preferred embodiment, the polymeric foam is formed from a silk solution containing butyric acid or salts, polymers, or derivatives thereof. The concentration and solvent type may be varied to control the pore sizes in the silk foam. In a preferred embodiment, the pores are sufficiently large to allow the in-growth of blood vessels and tissue when the silk foam is an implant.

The foams may also be produced by particulate leaching methods. Pore size and density can be controlled by selection of the leachable material, its size and quantity. Foams may be formed by dispersing particles in a solution of a permanent or resorbable polymer described above containing butyric acid or salts, polymers, or derivatives thereof, wherein the particles do not dissolve in the solvent. The solvent is subsequently evaporated, and the particles leached away with a solvent that dissolves just the particles. In a preferred embodiment, the foam is made from silk, and contains pores for cell, tissue and blood vessel in-growth if used as an implant.

The foams may also be produced by melt-foaming using blowing agents. The butyric acid or salts, polymers, or derivatives thereof may be blended with a permanent or resorbable polymer described above, and extruded with a blowing agent to form a foam for wound healing. In a preferred embodiment, the butyric acid or salts, polymers, or derivatives thereof are blended with resorbable polyesters, the blend is heated above its melt temperature, and a blowing agent added to form a foamable melt. The foamable melt is extruded through a die to form a foam comprising butyric acid or salts, polymers, or derivatives thereof for wound healing.

D. Controlled Release Wound Healing Devices Manufactured from Gels

The devices may contain gels of the permanent and/or resorbable polymers described above containing butyric acid or salts, polymers, or derivatives thereof. The gels may be used in dressings or implants.

In one preferred embodiment, the gels are made from the aqueous silk solutions described above containing butyric acid or salts, polymers, or derivatives thereof. These solutions may be gelled, for example, by vortexing, sonication, application of direct electrical current, by lowering the pH, or through chemical cross-linking

E. Controlled Release Wound Healing Devices Manufactured from Spheres and Other Particles

In still a further embodiment, the devices may be or contain spheres or particles, including micro- and nano-spheres that encapsulate butyric acid or salts, polymers, or derivatives thereof. These spheres or particles may be produced, for example, by emulsion-solvent evaporation, evaporation/extraction, phase separation/coacervation, self-assembly, solvent displacement, rapid expansion of supercritical solutions, spray drying or microfluidization.

In an embodiment, an aqueous solution of silk and butyric acid or salts, polymers, or derivatives thereof is added to a lipid dissolved in a solvent such as chloroform to form silk microspheres. After emulsification, the microspheres are subject to freeze/thaw cycles, lyophilized, re-suspended in alcohol, and centrifuged to remove the suspended lipid. In an alternative embodiment, an aqueous solution of silk and butyric acid or salts, polymers, or derivatives thereof is added to a solution of polyvinyl alcohol (PVA), and cast to faun a PVA film containing silk microspheres encapsulating butyric acid or salts, polymers, or derivatives thereof. The PVA is removed by dissolution.

In another embodiment, silk particles comprising butyric acid or salts, polymers, or derivatives thereof, are prepared from silk solutions (prepared as described above) by precipitating the particles from the silk solutions with organic solvents. Suitable solvents include acetone, methanol, ethanol, isopropanol, and butanol. Acetone and isopropanol are particularly preferred solvents. After precipitation, the particles may be collected by centrifugation, and dried. The sizes of the particles may be selected by choice of: (i) solvent, (ii) concentration of the silk solution, and (iii) the ratio of the solvent to the silk solution.

The particles comprising butyric acid or salts, polymers, or derivatives thereof may be applied directly to chronic wounds or incorporated into dressings that are applied to chronic wounds.

F. Controlled Release Wound Healing Devices Manufactured from Core-Sheath Fibers or Particulates

The devices may contain core-sheath fibers (or coaxial fibers), wherein the core-sheath fibers incorporate, and are configured for the controlled release of, butyric acid or salts, polymers, or derivatives thereof. The core-sheath fibers may be knit or woven into a device for wound healing, or may be incorporated as a component into a device for wound healing. For example, the core-sheath fibers may be incorporated into other fabric structures, such as non-wovens or woven textiles, films, foams, sponges, and gels. In one preferred embodiment, the core-sheath fibers are incorporated into porous implants, wherein the implants have pores large enough to allow cell, tissue and blood vessel in-growth.

In a preferred embodiment, the core of the core-sheath fibers is loaded with butyric acid or salts, polymers, or derivatives thereof. In a particularly preferred embodiment, the butyric acid or salts, polymers, or derivatives thereof are loaded in a polymer carrier that serves as the core. The polymer carrier may be a permanent or resorbable polymer, but preferably a polymer that allows a high loading of butyric acid or salts, polymers, or derivatives thereof in solid (e.g. powder) or solution form. Resorbable polymers are preferred if the devices are used as implants for wound healing. In a preferred embodiment, the core polymer carrier is a water-soluble polymer, a hydrogel, or a resorbable polymer. Examples of preferred polymer carriers for the core-sheath fiber include carboxymethyl cellulose, silk, collagen, and resorbable polyesters. The core structure may or may not have structural integrity. In the latter case, the sheath will provide the core-sheath structure with mechanical integrity. The core may, for example, be a polymeric monofilament or multifilament fiber containing butyric acid or salts, polymers, or derivatives thereof, or it may have the consistency of a powder, gel, slurry, paste, beads, etc.

The sheath of the core-sheath structure is configured to protect the core, provide storage stability, and in most instances to control the rate of release of the butyric acid or salts, polymers, or derivatives thereof. The sheath may also dictate the physical properties of the core-sheath structure, particularly if the core does not have structural integrity. The sheath may be made from the same polymeric material as the core, or one or more different materials. The sheath may be made from a permanent or resorbable polymer. However, it is preferably made from a resorbable polymer if the device is an implant. In order for the sheath-core structure to be able to deliver butyric acid or salts, polymers, or derivatives thereof, the sheath must be either permeable to these compounds, or it must degrade so these compounds can be released. In one preferred embodiment, the sheath is made from a resorbable polymer that allows the butyric acid or salts, polymers, or derivatives thereof to be released from the core as the resorbable polymer degrades as well as by diffusion through the resorbable polymer. Preferred resorbable polymers for the sheath include resorbable polyesters, silk, and collagen.

The rate of the release of the butyric acid or salts, polymers, or derivatives thereof from the core-sheath structure may be controlled by selection of at least the following: (i) form of the butyric acid (i.e. free acid, salt, polymer, or derivative), (ii) the choice of the core carrier material(s), (iii) the choice of the sheath material(s), (iv) the dimensions of the core and sheath, (v) crystallinity of the core and sheath structures, and (vi) the incorporation of porogens or other additives into the sheath material or polymer core carrier. In an embodiment, the core or sheath may contain one or more porogens to increase the rate of release of the butyric acid or salts, polymers, or derivatives thereof. An example of a porogen that may be incorporated into the sheath is calcium carbonate. The core or sheath may also incorporate additives, such as nucleants, plasticizers, surfactants, and/or buffers to control pH. In a preferred embodiment, the core-sheath structures deliver butyric acid or salts, polymers, or derivatives thereof to the wound for at least 3 days, more preferably at least 7 days, and even more preferably at least 14 days following placement on or in the wound.

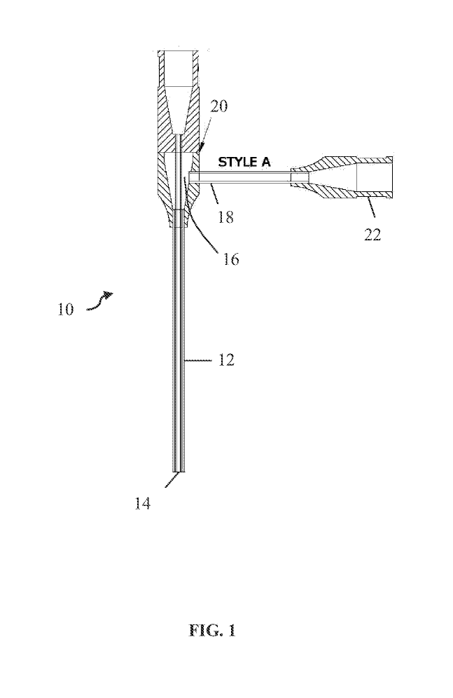

In one embodiment, the core-sheath structures may be manufactured using the device 10 of FIG. 1 by: (i) dissolving the core materials in a first solvent, (ii) dissolving the sheath materials in a second solvent, (iii) introducing the solution of the core materials into an inner needle 12 of a coaxial needle, (iv) introducing the solution of the sheath materials into an outer needle 18 of the coaxial needle shown in FIG. 1, and (v) spinning the solutions of the core and sheath materials through the coaxial needle and into a non-solvent. (In FIG. 1, the sheath material is delivered through the horizontal port 16, and core material is delivered through the vertical port 14.) The first and second solvents may be the same or different.

When the sheath material is a resorbable polyester, the preferred second solvents include chloroform and methylene chloride, and the preferred non-solvent is an alcohol such as isopropanol or hexane. Acetone is also a preferred second solvent when the resorbable polyester is soluble in acetone.

When the sheath material is silk, the preferred second solvent is water (i.e. an aqueous silk solution is used to make the sheath), and the preferred non-solvent is isopropanol, butanol, ethanol, methanol or acetone. Methanol and ethanol are preferred solvents when it is desirable to produce more crystalline core structures, and other solvents are preferred when it is desirable for the silk to be less crystalline.

The core-sheath fibers may be collected continuously from the non-solvent using, for example, a winder. Alternatively, the core-sheath fibers may be disrupted, for example, by agitation or manipulation of surface tension in order to collect short fibers, droplets, or particles. The rate of precipitation of the core-sheath fiber may be controlled by, for example, selection of the solvent system, size and geometry of the coaxial needle including the diameters of the inner and outer needles, use of different temperatures, use of surfactants and other additives, and the use of co-solvents in combination with the first, second, and non-solvent.

G. Controlled Release Wound Healing Devices Manufactured from Laminates

The devices may contain laminate structures configured for the controlled release of, butyric acid or salts, polymers, or derivatives thereof. The laminates may be used as thin films, perforated, or otherwise incorporated into devices for wound healing. Suitable laminates may be formed by compression molding a composition containing butyric acid or salts, polymers, or derivatives thereof between two sheets of a permanent or resorbable polymer. Preferred resorbable polymers include resorbable polyesters and silk. The composition containing butyric acid or salts, polymers, or derivatives thereof may further include other polymeric materials, as well as one or more additives. Preferably the composition containing butyric acid or salts, polymers, or derivatives thereof is in the form of a film or structure with mechanical integrity; however, it may also be in the form of a slurry, paste, paint or other material without structural integrity.

In one preferred embodiment, the laminate is perforated to allow cell, tissue and blood vessel in-growth, and used as an implant. In another preferred embodiment, the laminate is used as a dressing.

### IV. Methods of Using the Wound Healing Devices, and Their Applications

The devices may be used as dressings for wound healing or they may be used as implants if at least part of the device is resorbable. In an embodiment, the devices are used for the treatment of wounds. In a preferred embodiment, the devices are used for the treatment of chronic wounds, including venous stasis ulcers, diabetic ulcers, pressure ulcers, burns, trauma wounds, and surgical wounds.

The devices are placed on or in a wound so that the butyric acid or salts, polymers, or derivatives thereof can enter the wound. The devices may incorporate adhesives to help keep the device in place, and/or the devices may be held in place by another wound dressing material. For example, the devices may be held in place using compression dressings, such as when the devices are used to treat venous stasis ulcers.

In a preferred embodiment, the devices contain pores suitable for in-growth of blood vessels, cells and tissue when the devices are used as implants.

The devices may be left in place in the wound if they are resorbable implants, and do not need to be removed from the wound. However, these implants may also incorporate, for example, a moisture barrier or protective barrier that does need to be removed leaving behind the remainder of the implant.

The devices may also be used as dressings and removed after a period of time or replaced after a short period of time. In an embodiment, dressings may be replaced or additional implants placed in the wound in order to maintain a delivery of butyric acid to the wound.

Modifications and variations of the devices, processes, and methods described herein will be obvious to those skilled in the art and are intended to come within the scope of the appended claims.

We claim:

1. A device for the treatment of a wound, wherein the device releases an effective amount to enhance healing of butyric acid or salts, polymers, or derivatives thereof, to the wound when the device is placed onto or into the wound.

2. The device of claim 1, wherein the device is an implant.

3. The device of claim 1, wherein the device is a dressing.

4. The device of claim 1, wherein the device comprises silk.

5. The device of claim 1, wherein the device comprises a resorbable polymer.

6. The device of claim 5, wherein the resorbable polymer is a resorbable polyester.

7. The device of claim 1, wherein the device comprises one or more structure selected from the group consisting of fiber, film, foam, sponge, gel, sphere, particle, core-sheath fiber, and laminate, and combinations thereof.

8. The device of claim 7 wherein the core-sheath fiber comprises butyric acid or salts, polymers, or derivatives thereof in the core of the fiber.

9. The device of claim 1 wherein the device further comprises one or more therapeutic, prophylactic or diagnostic agents or additives.

10. The device of claim 9 wherein the agent is an inhibitor of metalloproteinases, an antibiotic, or a biofilm inhibitor.

11. The device of claim 1, wherein the device includes up to about 20% by weight of butyric acid or salts, polymers, or derivatives thereof.

12. The device of claim 1 wherein the device releases butyric acid or salts, polymers, or derivatives thereof to the wound for at least 3 days.

13. The device of claim 2 wherein the device has a sufficient porosity to allow the in-growth of blood vessels.

14. A medical implant comprising a silver salt of butyric acid.

15. A method of forming the device of claim 1, the method selected from one or more of the following: fiber spinning, film forming, foaming, gelling, core-sheath extrusion, lamination, sphere or particle encapsulation.

16. The method of claim 15 wherein the device comprises a resorbable polymer.

17. The method of claim 16 wherein the resorbable polymer is a resorbable polyester or silk.

18. A method of using the devices of claim 1, wherein the devices are implanted in a wound or applied to the surface of a wound.

19. The method of claim 18 wherein the wound is a venous stasis ulcer, pressure ulcer, diabetic ulcer, burn, trauma wound, or a surgical wound.
